(19) United States
(12) Patent Application Publication
(10) Pub. No.: US 2006/0057613 A1
(43) Pub. Date: Mar. 16, 2006

(54) Method for distinguishing methicillin resistant S. aureus from methicillin sensitive S. aureus in a mixed culture

(75) Inventors: Ramesh Ramakrishnan, San Jose, CA (US); Peter V. Riccelli, Tinley Park, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Nanosphere, Inc.

(21) Appl. No.: 11/189,546

(22) Filed: Jul. 26, 2005

Related U.S. Application Data
(60) Provisional application No. 60/591,127, filed on Jul. 26, 2004.

(51) Int. Cl. C12Q 1/68; C07H 21/04

(52) U.S. Cl. 435/6; 536/23.7

(57) ABSTRACT

The present invention provides isolated oligonucleotides and methods for detecting a methicillin resistant Staphylococcus aureus in a sample, including a sample that comprises nucleic acid molecules of higher biological complexity than that of amplified nucleic acid molecules.

This application claims the benefit of provisional application No. 60/591,127, filed Jul. 26, 2004.

### FIELD OF THE INVENTION

The invention relates to oligonucleotides and methods for detection of a methicillin resistant Staphylococcus aureus (MRSA) in a sample, including a sample that comprises nucleic acid molecules of higher biological complexity than that of amplified nucleic acid molecules, for example in genomic DNA.

### BACKGROUND OF THE INVENTION

Methicillin resistant strains of Staphylococcus aureus (MRSA) have become first ranking nosocomial pathogens worldwide. These bacteria are responsible for over 40% of all hospital-born staphylococcal infections in large teaching hospitals in the United States. Most recently they have become prevalent in smaller hospitals (20% incidence in hospitals with 200 to 500 beds), as well as in nursing homes (Wenzel et al., 1992, Am. J. Med. 91(Supp 3B):221-7). An unusual and most unfortunate property of MRSA strains is their ability to pick up additional resistance factors which suppress the susceptibility of these strains to other, chemotherapeutically useful antibiotics. Such multi-resistant strains of bacteria are now prevalent all over the world and the most “advanced” forms of these pathogens carry resistance mechanisms to most of the usable antibacterial agents (Blumberg et al., 1991, J. Inf. Disease, Vol. 63, pp. 1279-85).

Methicillin resistance is associated with the mecA gene. The gene is found on a piece of DNA of unknown, non-staphylococcal origin that the ancestral MRSA cell(s) probably acquired from a foreign source, and is referred to as the SCCmec element (Staphylococcal Cassette Chromosome mec; Ito et al., 2001, Agents Chemother. 45:1323-1336). The mecA gene encodes for a penicillin binding protein (PBP) called PBP2A (Murakami and Tomasz, 1989, J. Bacteriol. Vol. 171, pp. 874-79), which has very low affinity for the entire family of beta lactam antibiotics. In the current view, PBP2A is a kind of “surrogate” cell wall synthesizing enzyme that can take over the vital task of cell wall synthesis in staphylococci when the normal complement of PBPs (the normal catalysts of wall synthesis) can no longer function because they have become fully inactivated by beta lactam antibiotic in the environment. The critical nature of the mecA gene and its gene product PBP2A for the antibiotic resistant phenotype was demonstrated by early transposon inactivation experiments in which the transposon Tn551 was maneuvered into the mecA gene. The result was a dramatic drop in resistance level from the minimum inhibitory concentration (MIC) value of 1600 ug/ml in the parental bacterium to the low value of about 4 ug/ml in the transposon mutant (Matthews and Tomasz, 1990, Antimicrobial Agents and Chemotherapy, Vol. 34, pp. 1777-9).

Staphylococcal infections acquired in hospital have become increasingly difficult to treat with the rise of antibiotic resistant strains, and the increasing number of infections caused by both coagulase positive and negative Staphylococcal species. Effective treatment of these infections is diminished by the lengthy time many tests require for the determination of species identification (speciation) and antibiotic resistance. With the rapid identification of both species and antibiotic resistance status, the course of patient treatment can be implemented earlier and with less use of broad-spectrum antibiotics. Accordingly, there is a need for a rapid, highly sensitive and selective method for identifying and distinguishing Staphylococci species/or and for mecA gene detection.

Typically, to detect MRSA in a patient, a nasal swab is taken from the patient and cultured repeatedly, both in order to speciate the infection, as well as to determine resistance or sensitivity to the most commonly used antibiotic, methicillin or derivatives. The typical time taken to make a definitive diagnosis from swab to final assay is between 24 to 48 hours, primarily because of the need for multiple rounds of culturing. The need for culturing could be obviated by developing an assay for identifying MRSA directly from a swab.

No technique has emerged as a standard method for reliably distinguishing MRSA from a mixed culture containing methicillin sensitive Staphylococcus aureus (MSSA), as well as opportunistic non-pathogenic bacteria containing the mecA gene, from a nasal swab from a patient. Huletsky et al. have developed a method of identifying MRSA using real-time polymerase chain reaction (PCR) with probes that hybridize to nucleic acid sequences of MRSA at the right extremity junction of the mecA insertion site (Huletsky et al., 2004, J. of Clin. Microbiol. 42:1875-84; PCT Publication No. WO 02/099034). However, as pointed out recently by Diekema et al. (2004, J. Clin. Microbiol. July: 2879-83), the use of PCR for detection of antimicrobial resistance is fraught with risk, including the possibility of inhibition of the amplification process because of the quality of the patient sample (Paule et al., 2003, J. Clin. Microbiol. 41:4805-4807).

Consequently, the development of a technique capable of distinguishing these two populations from a mixed culture, such as a nasal swab, without PCR, would eliminate the false positive rate of MRSA calls, eliminate the need for administering methicillin for some patients, permit the clinician/doctor to administer alternate antibiotics (such as vancomycin), as well as shorten the hospital stay of the patient by eliminating 24-48 hours.

### SUMMARY OF THE INVENTION

The invention provides methods for detecting a methicillin resistant Staphylococcus aureus (MRSA) in a sample, wherein the sample comprises nucleic acid molecules of higher biological complexity than that of amplified nucleic acid molecules. The mecA gene is carried by a genetic element referred known as staphylococcal cassette chromosome mec (SCCmec) (Ito et al., 2001, Antimicrob. Agents Chemother. 45:1323-1336). The site of insertion of this mecA gene cassette into the Staphylococcus aureus genome is known and the sequence conserved (Ito et al., 2001, Antimicrob. Agents Chemother. 45:1323-1336). After insertion into the Staphylococcus aureus chromosome, the SCCmec has a left extremity junction region and a right extremity junction region (FIG. 1), where the SCCmec sequence is contiguous with the Staphylococcus aureus chromosomal sequence. In one aspect of the invention, the MRSA is detected with oligonucleotide probes having sequences that are complementary to the left junction of the mecA gene cassette insertion site, including part of the mecA gene cassette sequence and part of the Staphylococcus aureus sequence in the region of insertion.

The invention provides isolated oligonucleotides consisting of: (a) a nucleic acid sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10; or (b) a nucleic acid sequence that hybridizes with the complement of the nucleic acid sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. The invention also provides vectors comprising an oligonucleotide of the invention, host cells comprising the vector of the invention, and kits comprising an isolated oligonucleotide of the invention.

In one aspect, the methods for detecting MRSA in a sample comprise the steps of: a) providing an addressable substrate having a capture oligonucleotide bound thereto, wherein the capture oligonucleotide has a sequence complementary to a portion of the mecA gene cassette at the left junction and a portion of the Staphylococcus aureus sequence at the region of insertion; b) providing a detection probe comprising detector oligonucleotides, wherein the detector oligonucleotides have sequences that are complementary to at least a portion of the MRSA nucleic acid sequence; c) contacting the sample with the substrate and the detection probe under conditions that are effective for the hybridization of the capture oligonucleotide to the MRSA nucleic acid sequence and the hybridization of the detection probe to the MRSA nucleic acid sequence; d) washing the substrate to remove non-specifically bound material; and e) detecting whether the capture oligonucleotide and detection probe hybridized with the MRSA nucleic acid sequence.

In another aspect, the methods for detecting a target nucleic acid sequence in a sample without prior target amplification or complexity reduction comprise the steps of: a) providing an addressable substrate having a capture oligonucleotide bound thereto, wherein the capture probe comprises an oligonucleotide having a sequence complementary to at least a portion of the MRSA nucleic acid sequence; b) providing a detection probe comprising detector oligonucleotides, wherein the detector oligonucleotides have sequences that are complementary to a portion of the mecA gene cassette at the left junction and a portion of the Staphylococcus aureus insertion site; c) contacting the sample with the substrate and the detection probe under conditions that are effective for the hybridization of the capture oligonucleotide to the MRSA nucleic acid sequence and the hybridization of the detection probe to the MRSA nucleic acid sequence; d) washing to the substrate to remove non-specifically bound material; and e) detecting whether the capture oligonucleotide and detection probe hybridized with the MRSA nucleic acid sequence.

In a particular aspect, a capture or detector oligonucleotide having a sequence complementary to a portion of the mecA gene cassette at the left junction and a portion of the Staphylococcus aureus insertion site comprises a sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In another particular aspect, a capture or detector oligonucleotide having a sequence complementary to at least a portion of the MRSA nucleic acid sequence comprises a nucleic acid sequence as set forth in SEQ ID NO: 11; SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23.

In another embodiment, the nucleic acid molecules in a sample can comprise genomic DNA, genomic RNA, expressed RNA, plasmid DNA, mitochondrial or other cell organelle DNA, free cellular DNA, viral DNA or viral RNA, or a mixture of two or more of the above.

In one embodiment, a substrate used in a method of the invention can comprise a plurality of capture oligonucleotides, each of which can recognize one or more different single nucleotide polymorphisms or nucleotide differences, and the sample can comprise more than one nucleic acid target, each of which comprises a different single nucleotide polymorphism or nucleotide difference that can hybridize with one of the plurality of capture oligonucleotides. In addition, one or more types of detector probes can be provided in a method of the invention, each of which has detector oligonucleotides bound thereto that are capable of hybridizing with a different nucleic acid target.

In one embodiment, a sample can be contacted with the detector probe so that a nucleic acid target present in the sample hybridizes with the detector oligonucleotides on the detector probe, and the nucleic acid target bound to the detector probe can then be contacted with the substrate so that the nucleic acid target hybridizes with the capture oligonucleotide on the substrate. Alternatively, a sample can be contacted with the substrate so that a nucleic acid target present in the sample hybridizes with a capture oligonucleotide, and the nucleic acid target bound to the capture oligonucleotide can then be contacted with the detector probe so that the nucleic acid target hybridizes with the detector oligonucleotides on the detector probe. In another embodiment, a sample can be contacted simultaneously with the detector probe and the substrate.

In yet another embodiment, a detector oligonucleotide can comprise a detectable label. The label can be, for example, fluorescent, luminescent, phosphorescent, radioactive, or a nanoparticle, and the detector oligonucleotide can be linked to a dendrimer, a molecular aggregate, a quantum dot, or a bead. The label can allow for detection, for example, by photonic, electronic, acoustic, opto-acoustic, gravity, electro-chemical, electro-optic, mass-spectrometric, enzymatic, chemical, biochemical, or physical means.

In one embodiment, the detector probe can be a nanoparticle probe having detector oligonucleotides bound thereto. The nanoparticles can be made of, for example, a noble metal, such as gold or silver. A nanoparticle can be detected, for example, using an optical or flatbed scanner. The scanner can be linked to a computer loaded with software capable of calculating grayscale measurements, and the grayscale measurements are calculated to provide a quantitative measure of the amount of nucleic acid detected. Where the nanoparticle is made of gold, silver, or another metal that can promote autometallography, the substrate that is bound to the nanoparticle by means of a target nucleic acid molecule can be detected with higher sensitivity using a signal amplification step, such as silver stain. Alternatively, the substrate bound to a nanoparticle can be detected by detecting light scattered by the nanoparticle using methods as described, for example, in U.S. Ser. No. 10/008,978, filed Dec. 7, 2001, PCT/US01/46418, filed Dec. 7, 2001, U.S. Ser. No. 10/854,848, filed May 27, 2004, U.S. Ser. No. 10/995,051, filed Nov. 22, 2004, PCT/US04/16656, filed May 27, 2004, all of which are hereby incorporated by reference in their entirety.

In another embodiment, oligonucleotides attached to a substrate can be located between two electrodes, the nanoparticles can be made of a material that is a conductor of electricity, and step (e) in the methods of the invention can comprise detecting a change in conductivity. The electrodes can be made, for example, of gold and the nanoparticles are made of gold. Alternatively, a substrate can be contacted with silver stain to produce a change in conductivity.

In certain embodiments, a capture probe and substrate can be bound by specific binding pair interactions. In other embodiments, a capture probe and substrate can comprise complements of a specific binding pair. Complements of a specific binding pair can comprise nucleic acid, oligonucleotide, peptide nucleic acid, polypeptide, antibody, antigen, carbohydrate, protein, peptide, amino acid, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances.

Specific preferred embodiments of the present invention will become evident from the following more detailed description of certain preferred embodiments and the claims.

### BRIEF DESCRIPTION OF THE DRAWINGS

FIG. 1 shows a diagram of the location of junction capture probes at the left junction of the mecA gene cassette insertion site in Staphylococcus aureus.

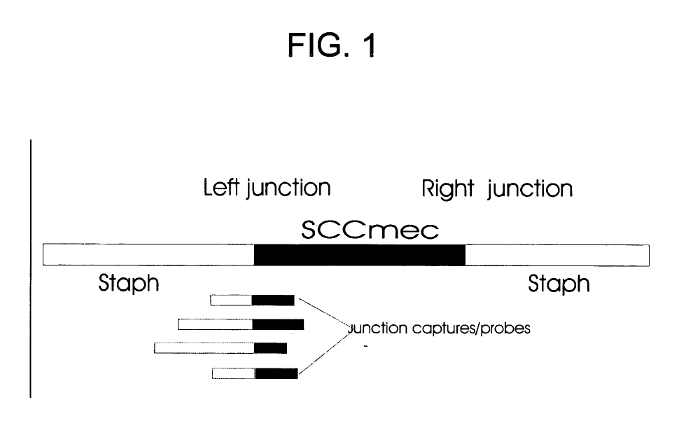

FIG. 2 shows a schematic representation of the single-step hybridization process of the invention.

FIG. 3 shows a schematic representation of the two-step hybridization process of the invention.

FIG. 4 illustrates schematically a hybridized complex of a nanoparticle-labeled detection probe, a wild-type or mutant capture probe bound to a substrate, and a wild-type target.

FIG. 5 shows results that demonstrate the extreme specificity of the junction capture/probe approach of the invention compared to a more conventional hybridization approach. DNA from a methicillin sensitive Staphylococcus aureus strain was deliberately spiked with various molar ratios of DNA from a methicillin resistant Staphylococcus epidermitis strain. The resulting DNA mixture was used to hybridize with a microarray slides containing specific left junction captures, along with a specific nanoparticle probe (NanoRR2), and the intensity results are shown in the upper panel. The lower panel shows the hybridization results when the same DNA mixture is hybridized to the mecA gene capture, while using a nanoparticle probe specific to the mecA gene. The results with the junction captures/probes show no cross-hybridization regardless of the amount of MRSE DNA present, whereas when the mecA gene specific capture/probe combination is used, extensive cross-hybridization is observed, even with extremely small amounts of spiked MRSE DNA.

### DETAILED DESCRIPTION OF THE PREFERRED EMBODIMENTS

Unless otherwise required by context, singular terms shall include pluralities and plural terms shall include the singular.

As utilized in accordance with the present disclosure, the following terms, unless otherwise indicated, shall be understood to have the following meanings:

As used herein, a “nucleic acid sequence,” a “nucleic acid molecule,” or “nucleic acids” refers to one or more oligonucleotides or polynucleotides as defined herein. As used herein, a “target nucleic acid molecule” or “target nucleic acid sequence” refers to an oligonucleotide or polynucleotide comprising a sequence that a user of a method of the invention desires to detect in a sample.

The term “polynucleotide” as referred to herein means a single-stranded or double-stranded nucleic acid polymer composed of multiple nucleotides. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “oligonucleotide” referred to herein includes naturally occurring, and modified nucleotides linked together by naturally occurring, and/or non-naturally occurring oligonucleotide linkages. Oligonucleotides are a polynucleotide subset comprising members that are generally single-stranded and have a length of 200 bases or fewer. In certain embodiments, oligonucleotides are 2 to 60 bases in length. In certain embodiments, oligonucleotides are 12, 13, 14, 15, 16, 17, 18, 19, 20, 21, 22, 23, 24, or 25 to 40 bases in length. In certain other embodiments, oligonucleotides are 25 or fewer bases in length. Oligonucleotides may be single stranded or double stranded, e.g. for use in the construction of a gene mutant. Oligonucleotides of the invention may be sense or antisense oligonucleotides with reference to a protein-coding sequence.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

In one embodiment, the invention provides nucleic acid molecules that are related to any of a nucleic acid molecule as shown in any of SEQ 1N NO: 1-23. As used herein, a “related nucleic acid molecule” includes allelic or splice variants of the nucleic acid molecule of any of SEQ ID NO: 1-23, and include sequences which are complementary to any of the above nucleotide sequences. In addition, related nucleic acid molecules also include those molecules which comprise nucleotide sequences which hybridize under moderately or highly stringent conditions as defined herein with the fully complementary sequence of the nucleic acid molecule of any of SEQ ID NO: 1-23, or of a nucleic acid fragment as defined herein. Hybridization probes may be prepared using the nucleotide sequences provided herein to screen cDNA, genomic or synthetic DNA libraries for related sequences. Regions of the nucleotide sequence of the nucleic acid molecules of the invention that exhibit significant identity to known sequences are readily determined using sequence alignment algorithms as described herein and those regions may be used to design probes for screening.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual (2nd ed., Cold Spring Harbor Laboratory, 1989); Anderson et al., Nucleic Acid Hybridisation: A Practical Approach Ch. 4 (IRL Press Limited).

More stringent conditions (such as higher temperature, lower ionic strength, higher formamide, or other denaturing agent) may also be used—however, the rate of hybridization will be affected. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄, (SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH. See Anderson et al., Nucleic Acid Hybridisation: A Practical Approach Ch. 4 (IRL Press Limited).

Factors affecting the stability of DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:
Tₘ(° C.)=81.5+16.6(log[Na+])+0.41(% G+C)−600/N−0.72(% formamide)
where N is the length of the duplex formed, [Na+] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, “moderately stringent conditions” of 50° C. in 0.015 M sodium ion will allow about a 21% mismatch.

It will be appreciated by those skilled in the art that there is no absolute distinction between “highly stringent conditions” and “moderately stringent conditions.” For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1M NaCl* for oligonucleotide probes up to about 20 nt is given by:
Tₘ=2° C. per A-T base pair+4° C. per G-C base pair
*The sodium ion concentration in 6× salt sodium citrate (SSC) is 1M. See Suggs et al., Developmental Biology Using Purified Genes 683 (Brown and Fox, eds., 1981).

High stringency washing conditions for oligonucleotides are usually at a temperature of 0-5° C. below the Tm of the oligonucleotide in 6×SSC, 0.1% SDS.

In another embodiment, related nucleic acid molecules comprise or consist of a nucleotide sequence that is at least about 70 percent identical to the nucleotide sequence as shown in any of SEQ ID NO: 1-23. In preferred embodiments, the nucleotide sequences are about 75 percent, or about 80 percent, or about 85 percent, or about 90 percent, or about 95, 96, 97, 98, or 99 percent identical to the nucleotide sequence as shown in any of SEQ ID NO: 1-23.

The term “identity,” as known in the art, refers to a relationship between the sequences of two or more polypeptide molecules or two or more nucleic acid molecules, as determined by comparing the sequences thereof. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “similarity” is used in the art with regard to a related concept, but in contrast to “identity,” “similarity” refers to a measure of relatedness, which includes both identical matches and conservative substitution matches. If two polypeptide sequences have, for example, 10/20 identical amino acids, and the remainder are all non-conservative substitutions, then the percent identity and similarity would both be 50%. If in the same example, there are five more positions where there are conservative substitutions, then the percent identity remains 50%, but the percent similarity would be 75% (15/20). Therefore, in cases where there are conservative substitutions, the percent similarity between two polypeptides will be higher than the percent identity between those two polypeptides.

Identity and similarity of related nucleic acids and polypeptides can be readily calculated by known methods. Such methods include, but are not limited to, those described in COMPUTATIONAL MOLECULAR BIOLOGY, (Lesk, A. M., ed.), 1988, Oxford University Press, New York; BIOCOMPUTING: INFORMATICS AND GENOME PROJECTS, (Smith, D. W., ed.), 1993, Academic Press, New York; COMPUTER ANALYSIS OF SEQUENCE DATA, Part 1, (Griffin, A. M., and Griffin, H. G., eds.), 1994, Humana Press, New Jersey; von Heinje, G., SEQUENCE ANALYSIS IN MOLECULAR BIOLOGY, 1987, Academic Press; SEQUENCE ANALYSIS PRIMER, (Gribskov, M. and Devereux, J., eds.), 1991, M. Stockton Press, New York; Carillo et al., 1988, SIAM J. Applied Math., 48:1073; and Durbin et al., 1998, BIOLOGICAL SEQUENCE ANALYSIS, Cambridge University Press.

Preferred methods to determine identity are designed to give the largest match between the sequences tested. Methods to determine identity are described in publicly available computer programs. Preferred computer program methods to determine identity between two sequences include, but are not limited to, the GCG program package, including GAP (Devereux et al., 1984, Nucl. Acid. Res., 12:387; Genetics Computer Group, University of Wisconsin, Madison, Wis.), BLASTP, BLASTN, and FASTA (Altschul et al., 1990, J. Mol. Biol., 215:403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990, supra). The well-known Smith Waterman algorithm may also be used to determine identity.

For example, using the computer algorithm GAP (Genetics Computer Group, University of Wisconsin, Madison, Wis.), two nucleic acid molecules for which the percent sequence identity is to be determined are aligned for optimal matching of their respective nucleotides (the “matched span,” as determined by the algorithm). A gap opening penalty (which is calculated as 3× the average diagonal; the “average diagonal” is the average of the diagonal of the comparison matrix being used; the “diagonal” is the score or number assigned to each perfect nucleotide match by the particular comparison matrix) and a gap extension penalty (which is usually 0.1× the gap opening penalty), as well as a comparison matrix such as PAM 250 or BLOSUM 62 are used in conjunction with the algorithm. A standard comparison matrix is also used by the algorithm (see Dayhoff et al., 5 Atlas of Protein Sequence and Structure (Supp. 3 1978)(PAM250 comparison matrix); Henikoff et al., 1992, Proc. Natl. Acad. Sci USA 89:10915-19 (BLOSUM 62 comparison matrix)).

Preferred parameters for nucleic acid molecule sequence comparison include the following:
Algorithm: Needleman and Wunsch, supra;
Comparison matrix: matches=+10, mismatch=0
Gap Penalty: 50
Gap Length Penalty: 3

The GAP program is also useful with the above parameters. The aforementioned parameters are the default parameters for nucleic acid molecule comparisons.

Other exemplary algorithms, gap opening penalties, gap extension penalties, comparison matrices, and thresholds of similarity may be used, including those set forth in the Program Manual, Wisconsin Package, Version 9, September, 1997. The particular choices to be made will be apparent to those of skill in the art and will depend on the specific comparison to be made, such as DNA-to-DNA, protein-to-protein, protein-to-DNA; and additionally, whether the comparison is between given pairs of sequences (in which case GAP or BestFit are generally preferred) or between one sequence and a large database of sequences (in which case FASTA or BLASTA are preferred).

The term “homology” refers to the degree of similarity between protein or nucleic acid sequences. Homology information is useful for the understanding the genetic relatedness of certain protein or nucleic acid species. Homology can be determined by aligning and comparing sequences. Typically, to determine amino acid homology, a protein sequence is compared to a database of known protein sequences. Homologous sequences share common functional identities somewhere along their sequences. A high degree of similarity or identity is usually indicative of homology, although a low degree of similarity or identity does not necessarily indicate lack of homology.

The nucleic acid molecules of the invention can readily be obtained in a variety of ways including, without limitation, chemical synthesis, cDNA or genomic library screening, expression library screening, and/or PCR amplification of cDNA.

Recombinant DNA methods used herein are generally those set forth in Sambrook et al., Molecular Cloning: A Laboratory Manual (Cold Spring Harbor Laboratory Press, 1989) and/or Current Protocols in Molecular Biology (Ausubel et al., eds., Green Publishers Inc. and Wiley and Sons 1994). The invention provides for nucleic acid molecules as described herein and methods for obtaining such molecules.

A “substrate” used in a method of the invention can be any surface capable of having oligonucleotides bound thereto. Such surfaces include, but are not limited to, glass, metal, plastic, or materials coated with a functional group designed for binding of oligonucleotides. The coating may be thicker than a monomolecular layer; in fact, the coating could involve porous materials of sufficient thickness to generate a porous 3-dimensional structure into which the oligonucleotides can diffuse and bind to the internal surfaces.

The term “addressable substrate” as used herein refers to a substrate that comprises one or more discrete regions, such as rows of spots, wherein each region or spot can contain a different type of oligonucleotide designed to bind to a portion of a target oligonucleotide. A sample containing one or more target oligonucleotides can be applied to each region or spot, and the rest of the assay can be performed in one of the ways described herein.

As used herein, a “type of oligonucleotides” refers to a plurality of oligonucleotide molecules having the same sequence. A “type of” nanoparticles, conjugates, particles, latex microspheres, etc. having oligonucleotides attached thereto refers to a plurality of that item having the same type(s) of oligonucleotides attached to them. “Nanoparticles having oligonucleotides attached thereto” are also sometimes referred to as “nanoparticle-oligonucleotide conjugates” or, in the case of the detection methods of the invention, “nanoparticle-oligonucleotide probes,” “nanoparticle probes,” or just “probes.”

The terms “bind” and “bound” and all grammatical variations thereof are used herein to refer to the ability of molecules to stick to each other because of the conformation and/or shape and chemical nature of parts of their surfaces. For example, enzymes can bind to their substrates; antibodies can bind to their antigens; and DNA strands can bind to their complementary strands. Binding can be characterized, for example, by a binding constant or association constant (Kₐ), or its inverse, the dissociation constant (K_{d}).

The term “complement” and grammatical variations thereof as used herein refers to nucleic acid sequences that form hydrogen bonds with each other at complementary nucleotide base pairs (i.e. adenine pairs with thymine in DNA or with uracil in RNA, and guanine pairs with cytosine). A “complement” can be one of a pair of portions or strands of a nucleic acid sequence that can hybridize with each other. A “complement” of a nucleic acid sequence as used herein does not necessarily have to have a complementary base pair at every position, but has a number of complementary base pairs sufficient to allow hybridization of the nucleic acid molecule to its complement under moderately and/or highly stringent conditions as described herein.

The term “capture oligonucleotide” as used herein refers to an oligonucleotide that is bound to a substrate and comprises a nucleic acid sequence that can locate (i.e. hybridize in a sample) a complementary nucleotide sequence or gene on a target nucleic acid molecule, thereby causing the target nucleic acid molecule to be attached to the substrate via the capture oligonucleotide upon hybridization. Suitable, but non-limiting examples of a capture oligonucleotide include DNA, RNA, PNA, LNA, or a combination thereof. The capture oligonucleotide may include natural sequences or synthetic sequences, with or without modified nucleotides.

A “detection probe” of the invention can be any carrier to which one or more detection oligonucleotides can be attached, wherein the one or more detection oligonucleotides comprise nucleotide sequences complementary to a particular nucleic acid sequence. The carrier itself may serve as a label, or may contain or be modified with a detectable label, or the detection oligonucleotides may carry such labels. Carriers that are suitable for the methods of the invention include, but are not limited to, nanoparticles, quantum dots, dendrimers, semi-conductors, beads, up- or down-converting phosphors, large proteins, lipids, carbohydrates, or any suitable inorganic or organic molecule of sufficient size, or a combination thereof.

As used herein, a “detector oligonucleotide” or “detection oligonucleotide” is an oligonucleotide as defined herein that comprises a nucleic acid sequence that can be used to locate (i.e. hybridize in a sample) a complementary nucleotide sequence or gene on a target nucleic acid molecule. Suitable, but non-limiting examples of a detection oligonucleotide include DNA, RNA, PNA, LNA, or a combination thereof. The detection oligonucleotide may include natural sequences or synthetic sequences, with or without modified nucleotides.

In one embodiment, a capture or detector oligonucleotide has a sequence complementary to a portion of the mecA gene cassette and a portion of the Staphylococcus aureus insertion site at the left side junction (i.e. the complementary sequence spans across the insertion site to hybridize mecA gene cassette sequence on one side and Staphylococcus aureus gene sequence on the other side of the insertion site). In a particular embodiment, such oligonucleotides comprise a sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

As used herein, the term “mecA gene cassette” refers to the genetic element as defined as SCCmec, which carries the mecA gene and is inserted into Staphylococcus aureus genome as described in Ito et al. (2001, Antimicrob. Agents Chemother. 45:1323-1336). As used herein, the “insertion site” is the site where the mecA gene cassette joins the Staphylococcus aureus genome, i.e. on one side of the insertion site is mecA gene cassette sequence and on the other side is Staphylococcus aureus sequence. The site of insertion is described in Ito et al. (2001, Antimicrob. Agents Chemother. 45:1323-1336) and in U.S. Pat. No. 6,156,507, which are incorporated by reference herein.

In another embodiment, a capture or detector oligonucleotide having a sequence complementary to at least a portion of the Staphylococcus aureus genomic nucleic acid sequence. In a particular embodiment, such oligonucleotides comprise a nucleic acid sequence as set forth in SEQ ID NO: 1; SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23.

As used herein, the terms “label” refers to a detectable marker that may be detected by photonic, electronic, opto-electronic, magnetic, gravity, acoustic, enzymatic, or other physical or chemical means. The term “labeled” refers to incorporation of such a detectable marker, e.g., by incorporation of a radiolabeled nucleotide or attachment to an oligonucleotide of a detectable marker.

A “sample” as used herein refers to any quantity of a substance that comprises nucleic acids and that can be used in a method of the invention. For example, the sample can be a biological sample or can be extracted from a biological sample derived from humans, animals, plants, fungi, yeast, bacteria, viruses, tissue cultures or viral cultures, or a combination of the above. They may contain or be extracted from solid tissues (e.g. bone marrow, lymph nodes, brain, skin), body fluids (e.g. serum, blood, urine, sputum, seminal or lymph fluids), skeletal tissues, or individual cells. Alternatively, the sample can comprise purified or partially purified nucleic acid molecules and, for example, buffers and/or reagents that are used to generate appropriate conditions for successfully performing a method of the invention.

In one embodiment of the invention, the target nucleic acid molecules in a sample can comprise genomic DNA, genomic RNA, expressed RNA, plasmid DNA, cellular nucleic acids or nucleic acids derived from cellular organelles (e.g. mitochondria) or parasites, or a combination thereof.

In another embodiment, target nucleic acid molecules in a sample can be amplified. Several methods for amplifying nucleic acid molecules are known in the art as described for example in Sambrook et al., 2001, MOLECULAR CLONING: A LABORATORY MANUAL, 3d ed., Cold Spring Harbor Laboratory Press, Cold Spring Harbor, N.Y., which is incorporated herein by reference for any purpose. Such methods include, for example, polymerase chain reaction (PCR), rolling circle amplification, and whole genomic amplification using degenerate primers. Additional exemplary methods include nucleic acid sequence based amplification (NASBA) and isothermal and chimeric primer-initiated amplification of nucleic acids (ICAN™, Takara Bio Inc, Japan). Those of skill in the art will recognize that NASBA is a transcription-based amplification method that amplifies RNA from either an RNA or DNA target, and can be executed using protocols available, for example, from bioMerieux (Boxtel, The Netherlands). Certain examples of PCR amplification of nucleic acid molecules useful in the methods of the invention are described, for example, in U.S. Pat. No. 5,629,156, U.S. Pat. No. 5,750,338, and U.S. Pat. No. 5,780,224, the disclosures of all of which are incorporated by reference.

As used herein, the “biological complexity” of a nucleic acid molecule refers to the number of non-repeat nucleotide sequences present in the nucleic acid molecule, as described, for example, in Lewin, GENE EXPRESSION 2, Second Edition: Eukaryotic Chromosomes, 1980, John Wiley & Sons, New York, which is hereby incorporated by reference. For example, a simple oligonucleotide of 30 bases that contains a non-repeat sequence has a complexity of 30. The E. coli genome, which contains 4,200,000 base pairs, has a complexity of 4,200,000, because it has essentially no repeat sequences. Bacterial genomes typically range from about 500,000 to about 10,000,000 base pairs (Casjens, 1998, Annu. Rev. Genet. 32:339-77), corresponding to complexities of about 500,000 to about 10,000,000, respectively. The genomes of the methicillin resistant Staphylococcus aureus MRSA252 has a genome of 2,902,619 base pairs (GenBank Accession No. NC_{—}002952), and the methicillin sensitive Staphylococcus aureus MSSA476 (GenBank Accession No. NC_{—}002953) has a genome of 2,799,802 base pairs. The Staphylococcus aureus genomes have few repeat sequences, and have overall complexities of about 3,000,000. The human genome, in contrast, has on the order of 3,000,000,000 base pairs, much of which is repeat sequences (e.g. about 2,000,000,000 base pairs). The overall complexity (i.e. number of non-repeat nucleotides) of the human genome is on the order of 1,000,000,000.

The complexity of a nucleic acid molecule, such as a DNA molecule, does not depend on a number of different repeat sequences (i.e. copies of each different sequence present in the nucleic acid molecule). For example, if a DNA has 1 sequence that is a nucleotides long, 5 copies of a sequence that is b nucleotides long, and 50 copies of a sequence that is c nucleotides long, the complexity will be a+b+c, while the repetition frequencies of sequence a will be 1, b will be 5, and c will be 10.

The total length of different sequences within a given DNA can be determined experimentally by calculating the C₀t_{1/2} for the DNA, which is represented by the following formula,

$${C}_{0} {t}_{1 \text{/} 2} = \frac{1}{k}$$

where C is the concentration of DNA that is single stranded at time t_{1/2} (when the reaction is ½ complete) and k is the rate constant. A C₀t_{1/2} represents the value required for half reassociation of two complementary strands of a DNA. Reassociation of DNA is typically represented in the form of Cot curves that plot the fraction of DNA remaining single stranded (C/C₀) or the reassociated fraction (1−C/C₀) against the log of the C₀t. Cot curves were introduced by Britten and Kohne in 1968 (1968, Science 161:529-540). Cot curves demonstrate that the concentration of each reassociating sequence determines the rate of renaturation for a given DNA. The C₀t_{1/2}, in contrast, represents the total length of different sequences present in a reaction.

The C₀t_{1/2} of a DNA is proportional to its complexity. Thus, determining the complexity of a DNA can be accomplished by comparing its C₀t_{1/2} with the C₀t_{1/2} of a standard DNA of known complexity. Usually, the standard DNA used to determine biological complexity of a DNA is an E. coli DNA, which has a complexity identical to the length of its genome (4.2×10⁶ base pairs) since every sequence in the E. coli genome is assumed to be unique. Therefore, the following formula can be used to determine biological complexity for a DNA.

$$\frac{{C}_{0} {t}_{1 \text{/} 2} \text{(any DNA)}}{{C}_{0} {t}_{1 \text{/} 2} ( E . coli DNA )} = \frac{\text{complexity(any DNA)}}{4.2 \times {10}^{6}}$$

In certain embodiments, the invention provides methods for reliable detection and discrimination (i.e. identification) of a methicillin resistant Staphylococcus aureus (MRSA) in a sample comprising genomic DNA without the need for enzymatic complexity reduction by PCR or any other method that preferentially amplifies a specific DNA sequence.

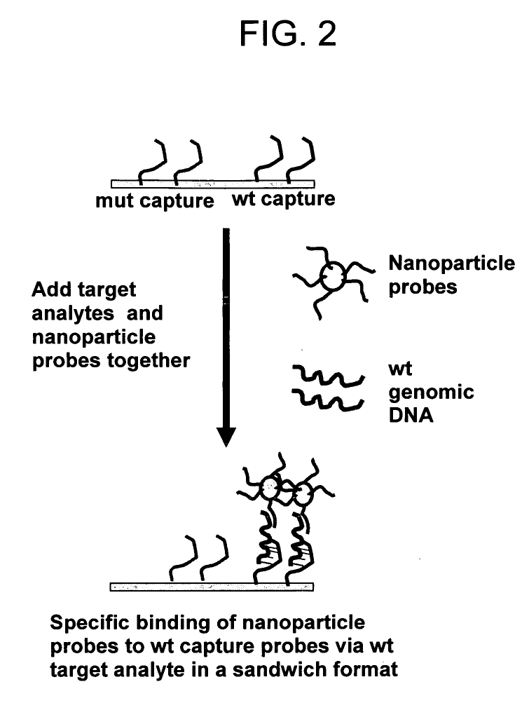

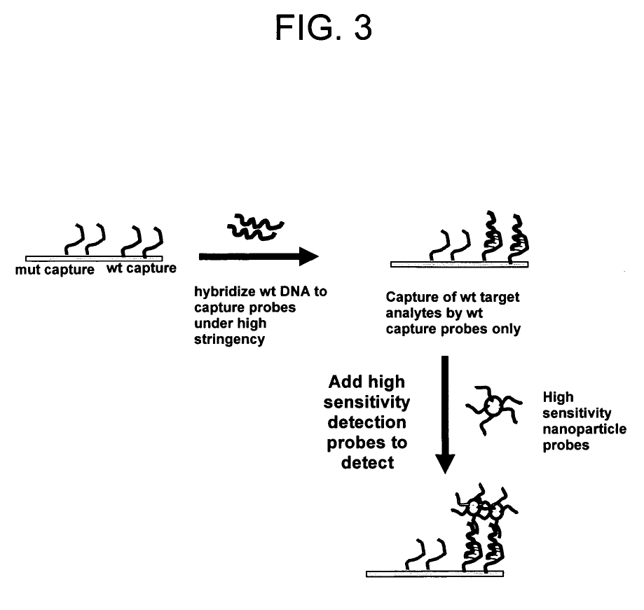

In one embodiment, the methods of the invention can be accomplished using a one-step or a two-step hybridization. FIG. 2 shows a schematic representation of the one-step hybridization. FIG. 3 shows a schematic representation of the two-step hybridization. In the two-step process, the hybridization events happen in two separate reactions. The target binds to the capture oligonucleotides first, and after removal of all non-bound nucleic acids, a second hybridization is performed that provides detection probes that can specifically bind to a second portion of the captured target nucleic acid.

Methods of the invention that involve the two-step hybridization will work without accommodating certain unique properties of the detection probes (such as high Tm and sharp melting behavior of nanoparticle probes) during the first hybridization event (i.e. capture of the target nucleic acid molecule) since the reaction occurs in two steps. Although the first step is not sufficiently stringent to capture only the desired target sequences, its application will result in considerable enrichment of the specific sequence of interest. Thus, the second step (binding of detection probes) is then provided to achieve the desired specificity for the target nucleic acid molecule. The combination of these two discriminating hybridization events allows the overall specificity for the target nucleic acid molecule. However, in order to achieve this exquisite specificity the hybridization conditions are chosen to be very stringent. Under such stringent conditions, only a small amount of target and detection probe gets captured by the capture probes. This amount of target is typically so small that it escapes detection by standard fluorescent methods because it is buried in the background. It is therefore critical for this invention to detect this small amount of target using an appropriately designed detection probe. The detection probes described in this invention consist in a carrier portion that is typically modified to contain many detection oligonucleotides, which enhances the hybridization kinetics of this detection probe. Second, the detection probe is also labeled with one or more high sensitivity label moieties, which together with the appropriate detection instrument, allows for the detection of the small number of captured target-detection probe complexes. Thus, it is the appropriate tuning of all factors in combination with a high sensitivity detection system that allows this process to work.

The two-step hybridization methods of the invention can comprise using any detection probes as described herein for the detection step. In a preferred embodiment, nanoparticle probes are used in the second step of the method. Where nanoparticles are used and the stringency conditions in the second hybridization step are equal to those in the first step, the detection oligonucleotides on the nanoparticle probes can be longer than the capture oligonucleotides. Thus, conditions necessary for the unique features of the nanoparticle probes (high Tm and sharp melting behavior) are not needed.

The single- and two-step hybridization methods in combination with the appropriately designed capture oligos and detection probes of the invention provide new and unexpected advantages over previous methods of detecting MRSA nucleic acid sequences in a sample. Specifically, the methods of the invention do not require an amplification step to maximize the number of targets and simultaneously reduce the relative concentration of non-target sequences in a sample to enhance the possibility of binding to the target, as required, for example, in polymerase chain reaction (PCR) based detection methods, nor does it require the use of radioactive tracers, which have their own inherent problems. Specific detection without prior target sequence amplification provides tremendous advantages. For example, amplification often leads to contamination of research or diagnostic labs, resulting in false positive test outcomes. PCR or other target amplifications require specifically trained personnel, costly enzymes and specialized equipment. Most importantly, the efficiency of amplification can vary with each target sequence and primer pair, leading to errors or failures in determining the target sequences and/or the relative amount of the target sequences present in a genome. In addition, the methods of the invention involve fewer steps and are thus easier and more efficient to perform than gel-based methods of detecting nucleic acid targets, such as Southern and Northern blot assays.

In one embodiment, the methods for detecting MRSA in a sample comprise the steps of: a) providing an addressable substrate having a capture oligonucleotide bound thereto, wherein the capture oligonucleotide has a sequence complementary to a portion of the mecA gene cassette and a portion of the Staphylococcus aureus insertion site at the left junction; b) providing a detection probe comprising detector oligonucleotides, wherein the detector oligonucleotides have sequences that are complementary to at least a portion of the MRSA nucleic acid sequence; c) contacting the sample with the substrate and the detection probe under conditions that are effective for the hybridization of the capture oligonucleotide to the MRSA nucleic acid sequence and the hybridization of the detection probe to the MRSA nucleic acid sequence; and d) detecting whether the capture oligonucleotide and detection probe hybridized with the MRSA nucleic acid sequence.

In another embodiment, the methods for detecting a target nucleic acid sequence in a sample without prior target amplification or complexity reduction comprise the steps of: a) providing an addressable substrate having a capture oligonucleotide bound thereto, wherein the capture oligonucleotide has a sequence complementary to at least a portion of the MRSA nucleic acid sequence; b) providing a detection probe comprising detector oligonucleotides, wherein the detector oligonucleotides have sequences that are complementary to a portion of the mecA gene gene cassette and a portion of the Staphylococcus aureus insertion site at the left junction; c) contacting the sample with the substrate and the detection probe under conditions that are effective for the hybridization of the capture oligonucleotide to the MRSA nucleic acid sequence and the hybridization of the detection probe to the MRSA nucleic acid sequence; and d) detecting whether the capture oligonucleotide and detection probe hybridized with the MRSA nucleic acid sequence.

In another embodiment, a detector oligonucleotide can be detectably labeled. Various methods of labeling polynucleotides are known in the art and may be used advantageously in the methods disclosed herein. In a particular embodiment, a detectable label of the invention can be fluorescent, luminescent, Raman active, phosphorescent, radioactive, or efficient in scattering light, have a unique mass, or other has some other easily and specifically detectable physical or chemical property, and in order to enhance said detectable property the label can be aggregated or can be attached in one or more copies to a carrier, such as a dendrimer, a molecular aggregate, a quantum dot, or a bead. The label can allow for detection, for example, by photonic, electronic, acoustic, opto-acoustic, gravity, electro-chemical, enzymatic, chemical, Raman, or mass-spectrometric means.

In one embodiment, a detector probe of the invention can be a nanoparticle probe having detector oligonucleotides bound thereto. Nanoparticles have been a subject of intense interest owing to their unique physical and chemical properties that stem from their size. Due to these properties, nanoparticles offer a promising pathway for the development of new types of biological sensors that are more sensitive, more specific, and more cost effective than conventional detection methods. Methods for synthesizing nanoparticles and methodologies for studying their resulting properties have been widely developed over the past 10 years (Klabunde, editor, Nanoscale Materials in Chemistry, Wiley Interscience, 2001). However, their use in biological sensing has been limited by the lack of robust methods for functionalizing nanoparticles with biological molecules of interest due to the inherent incompatibilities of these two disparate materials. A highly effective method for functionalizing nanoparticles with modified oligonucleotides has been developed. See U.S. Pat. Nos. 6,361,944 and 6,417,340 (assignee: Nanosphere, Inc.), which are incorporated by reference in their entirety. The process leads to nanoparticles that are heavily functionalized with oligonucleotides, which have surprising particle stability and hybridization properties. The resulting DNA-modified particles have also proven to be very robust as evidenced by their stability in solutions containing elevated electrolyte concentrations, stability towards centrifugation or freezing, and thermal stability when repeatedly heated and cooled. This loading process also is controllable and adaptable. Nanoparticles of differing size and composition have been functionalized, and the loading of oligonucleotide recognition sequences onto the nanoparticle can be controlled via the loading process. Suitable, but non-limiting examples of nanoparticles include those described U.S. Pat. No. 6,506,564; International Patent Application No. PCT/US02/16382; U.S. patent application Ser. No. 10/431,341 filed May 7, 2003; and International Patent Application No. PCT/US03/14100; all of which are hereby incorporated by reference in their entirety.

The aforementioned loading method for preparing DNA-modified nanoparticles, particularly DNA-modified gold nanoparticle probes, has led to the development of a new calorimetric sensing scheme for oligonucleotides. This method is based on the hybridization of two gold nanoparticle probes to two distinct regions of a DNA target of interest. Since each of the probes are functionalized with multiple oligonucleotides bearing the same sequence, the binding of the target results in the formation of target DNA/gold nanoparticle probe aggregate when sufficient target is present. The DNA target recognition results in a calorimetric transition due to the decrease in interparticle distance of the particles. This colorimetric change can be monitored optically, with a UV-vis spectrophotometer, or visually with the naked eye. In addition, the color is intensified when the solutions are concentrated onto a membrane. Therefore, a simple calorimetric transition provides evidence for the presence or absence of a specific DNA sequence. Using this assay, femtomole quantities and nanomolar concentrations of model DNA targets and polymerase chain reaction (PCR) amplified nucleic acid sequences have been detected, as well as with genomic DNA (Storhoff et al., 2004, Nature Biotechnology 22:883-7). Importantly, it has been demonstrated that gold probe/DNA target complexes exhibit extremely sharp melting transitions which makes them highly specific labels for DNA targets. In a model system, one base insertions, deletions, or mismatches were easily detectable via the spot test based on color and temperature, or by monitoring the melting transitions of the aggregates spectrophotometrically (Storhoff et. al, 1998, J. Am. Chem. Soc. 120:1959). See also, for instance, U.S. Pat. No. 5,506,564.

Due to the sharp melting transitions, the perfectly matched target could be detected even in the presence of the mismatched targets when the hybridization and detection was performed under extremely high stringency (e.g., a single degree below the melting temperature of the perfect probe/target match). It is important to note that with broader melting transitions such as those observed with molecular fluorophore labels, hybridization and detection at a temperature close to the melting temperature would result in significant loss of signal due to partial melting of the probe/target complex leading to lower sensitivity, and also partial hybridization of the mismatched probe/target complexes leading to lower specificity due to mismatched probe signal. Therefore, nanoparticle probes offer higher specificity detection for nucleic acid detection method.

As described herein, nanoparticle probes, particularly gold nanoparticle probes, are surprising and unexpectedly suited for direct detection of MRSA in a sample with genomic, bacterial DNA with or without amplification. First, the extremely sharp melting transitions observed in nanoparticle oligonucleotide detection probe translate to a surprising and unprecedented assay specificity that allows single base discrimination even in a human genomic DNA background. Second, a silver-based signal amplification procedure in a DNA microarray-based assay can further provide ultra-high sensitivity enhancement.

A nanoparticle can be detected in a method of the invention, for example, using an optical or flatbed scanner. The scanner can be linked to a computer loaded with software capable of calculating grayscale measurements, and the grayscale measurements are calculated to provide a quantitative measure of the amount of nucleic acid detected.

Suitable scanners include those used to scan documents into a computer which are capable of operating in the reflective mode (e.g., a flatbed scanner), other devices capable of performing this function or which utilize the same type of optics, any type of greyscale-sensitive measurement device, and standard scanners which have been modified to scan substrates according to the invention (e.g., a flatbed scanner modified to include a holder for the substrate) (to date, it has not been found possible to use scanners operating in the transmissive mode). The resolution of the scanner must be sufficient so that the reaction area on the substrate is larger than a single pixel of the scanner. The scanner can be used with any substrate, provided that the detectable change produced by the assay can be observed against the substrate (e.g., a gray spot, such as that produced by silver staining, can be observed against a white background, but cannot be observed against a gray background). The scanner can be a black-and-white scanner or, preferably, a color scanner.

Most preferably, the scanner is a standard color scanner of the type used to scan documents into computers. Such scanners are inexpensive and readily available commercially. For instance, an Epson Expression 636 (600×600 dpi), a UMAX Astra 1200 (300×300 dpi), or a Microtec 1600 (1600×1600 dpi) can be used. The scanner is linked to a computer loaded with software for processing the images obtained by scanning the substrate. The software can be standard software which is readily available commercially, such as Adobe Photoshop 5.2 and Corel Photopaint 8.0. Using the software to calculate greyscale measurements provides a means of quantitating the results of the assays.

The software can also provide a color number for colored spots and can generate images (e.g., printouts) of the scans, which can be reviewed to provide a qualitative determination of the presence of a nucleic acid, the quantity of a nucleic acid, or both. In addition, it has been found that the sensitivity of assays can be increased by subtracting the color that represents a negative result from the color that represents a positive result.

The computer can be a standard personal computer, which is readily available commercially. Thus, the use of a standard scanner linked to a standard computer loaded with standard software can provide a convenient, easy, inexpensive means of detecting and quantitating nucleic acids when the assays are performed on substrates. The scans can also be stored in the computer to maintain a record of the results for further reference or use. Of course, more sophisticated instruments and software can be used, if desired.

Silver staining can be employed with any type of nanoparticles that catalyze the reduction of silver. Preferred are nanoparticles made of noble metals (e.g., gold and silver). See Bassell, et al., J. Cell Biol., 126, 863-876 (1994); Braun-Howland et al., Biotechniques, 13, 928-931 (1992). If the nanoparticles being employed for the detection of a nucleic acid do not catalyze the reduction of silver, then silver ions can be complexed to the nucleic acid to catalyze the reduction. See Braun et al., Nature, 391, 775 (1998). Also, silver stains are known which can react with the phosphate groups on nucleic acids.

Silver staining can be used to produce or enhance a detectable change in any assay performed on a substrate, including those described above. In particular, silver staining has been found to provide a huge increase in sensitivity for assays employing a single type of nanoparticle so that the use of layers of nanoparticles, aggregate probes and core probes can often be eliminated.

In another embodiment, oligonucleotides attached to a substrate can be located between two electrodes, the nanoparticles can be made of a material that is a conductor of electricity, and step (d) in the methods of the invention can comprise detecting a change in conductivity. In yet another embodiment, a plurality of oligonucleotides, each of which can recognize a different target nucleic acid sequence, are attached to a substrate in an array of spots and each spot of oligonucleotides is located between two electrodes, the nanoparticles are made of a material that is a conductor of electricity, and step (d) in the methods of the invention comprises detecting a change in conductivity. The electrodes can be made, for example, of gold and the nanoparticles are made of gold. Alternatively, a substrate can be contacted with silver stain to produce a change in conductivity.

In a particular embodiment, nucleic acid molecules in a sample are of higher biological complexity than amplified nucleic acid molecules. One of skill in the art can readily determine the biological complexity of a target nucleic acid sequence using methods as described, for example, in Lewin, GENE EXPRESSION 2, Second Edition: Eukaryotic Chromosomes, 1980, John Wiley & Sons, New York, which is hereby incorporated by reference.

Hybridization kinetics are absolutely dependent on the concentration of the reaction partners, i.e. the strands that have to hybridize. In a given quantity of DNA that has been extracted from a cell sample, the amount of total genomic, mitochondrial (if present), and extra-chromosomal elements (if present) DNA is only a few micrograms. Thus, the actual concentrations of the reaction partners that are to hybridize will depend on the size of these reaction partners and the complexity of the extracted DNA. For example, a target sequence of 30 bases that is present in one copy per single genome is present in different concentrations when comparing samples of DNA from different sources and with different complexities. For example, the concentration of the same target sequence in 1 microgram of total human DNA is about 1000 fold lower than in a 1 microgram bacterial DNA sample, and it would be about 1,000,000 fold lower than in a sample consisting in 1 microgram of a small plasmid DNA.

In one embodiment, the hybridization conditions are effective for the specific and selective hybridization, whereby single base mismatches are detectable, of the capture oligonucleotide and/or the detector oligonucleotides to the target nucleic acid sequence, even when said target nucleic acid is part of a nucleic acid sample with a biological complexity of 50,000 or larger, as shown, for example, in the Examples below.

The methods of the invention can further be used for identifying specific species of a biological microorganism (e.g. Staphylococcus) and/or for detecting genes that confer antibiotic resistance (e.g. mecA gene which confers resistance to the antibiotic methicillin).

In another embodiment, the invention provides oligonucleotide sequences that bind a portion of the mecA gene cassette and the insertion site of the Staphylococcus aureus comprising the mecA gene at the left junction, and kits that employ these sequences. These sequences have been designed to be highly sensitive as well as selective for Staphylococcal species or the mecA gene, which gives rise to some forms of antibiotic resistance.

The invention also relates to a kit comprising at least one oligonucleotide that comprises a sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 and other reagents useful for detecting a methicillin resistant Staphylococcus aureus (MRSA) in biological samples. Such reagents may include a detectable label, blocking serum, positive and negative control samples, and detection reagents.

### EXAMPLES

The invention is demonstrated further by the following illustrative examples. The examples are offered by way of illustration and are not intended to limit the invention in any manner. In these examples all percentages are by weight if for solids and by volume if for liquids, and all temperatures are in degrees Celsius unless otherwise noted.

### Example 1

Single-Step and Two-Step Hybridization Methods for Identifying SNPs in Unamplified Genomic DNA Using Nanoparticle Probes

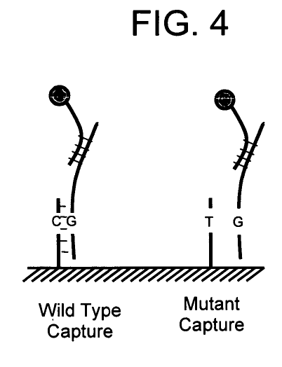

Gold nanoparticle-oligonucleotide probes to detect the target methicillin resistant Staphylococcus aureus (MRSA) sequences were prepared using procedures described in PCT/US97/12783, filed Jul. 21, 1997; PCT/US00/17507, filed Jun. 26, 2000; PCT/US01/01190, filed Jan. 12, 2001, which are incorporated by reference in their entirety. FIG. 4 illustrates conceptually the use of gold nanoparticle probes having oligonucleotides bound thereto for detection of target DNA using a DNA microarray having MRSA (methicillin resistant staph aureus) or MSSA (methicillin sensitive staph aureus) capture probe oligonucleotides. The sequence of the oligonucleotides bound to the nanoparticles are complementary to one portion of the sequence of target while the sequence of the capture oligonucleotides bound to the substrate are complementary to another portion of the target sequence. Under hybridization conditions, the nanoparticle probes, the capture probes, and the target sequence bind to form a complex. Signal detection of the resulting complex can be enhanced with conventional silver staining.

(a) Preparation Of Gold Nanoparticles

Gold colloids (13 nm diameter) were prepared by reduction of HAuCl₄ with citrate as described in Frens, 1973, Nature Phys. Sci., 241:20 and Grabar, 1995, Anal. Chem. 67:735. Briefly, all glassware was cleaned in aqua regia (3 parts HCl, 1 part HNO₃), rinsed with Nanopure H₂O, then oven dried prior to use. HAuCl₄ and sodium citrate were purchased from Aldrich Chemical Company. Aqueous HAuCl₄ (1 mM, 500 mL) was brought to reflux while stirring. Then, 38.8 mM sodium citrate (50 mL) was added quickly. The solution color changed from pale yellow to burgundy, and refluxing was continued for 15 min. After cooling to room temperature, the red solution was filtered through a Micron Separations Inc. 1 micron filter. Au colloids were characterized by UV-vis spectroscopy using a Hewlett Packard 8452A diode array spectrophotometer and by Transmission Electron Microscopy (TEM) using a Hitachi 8100 transmission electron microscope. Gold particles with diameters of 15 nm will produce a visible color change when aggregated with target and probe oligonucleotide sequences in the 10-35 nucleotide range.

(b) Synthesis Of Oligonucleotides

The capture probe oligonucleotides, which were designed to be complementary to specific target segments of the MRSA DNA sequence, were synthesized on a 1 micromole scale using a ABI 8909 DNA synthesizer in single column mode using phosphoramidite chemistry [Eckstein, F. (ed.) Oligonucleotides and Analogues: A Practical Approach (IRL Press, Oxford, 1991)]. The capture sequences contained either a 3′-amino modifier that serves as the active group for covalent attachment to the substrate during the arraying process. The oligonucleotides were synthesized by following standard protocols for DNA synthesis. Columns with the 3′-amino modifier attached to the solid support, the standard nucleotide phosphoramidites and reagents were obtained from Glen Research (Sterling, Va.). The final dimethoxytrityl (DMT) protecting group was not cleaved from the oligonucleotides to aid in purification. After synthesis, DNA was cleaved from the solid support using aqueous ammonia, resulting in the generation of a DNA molecule containing a free amine at the 3′-end. Reverse phase HPLC was performed with an Agilent 1100 series instrument equipped with a reverse phase column (Vydac) by using 0.03 M Et₃NH⁺ OAc⁻ buffer (TEAA), pH 7, with a 1%/min. gradient of 95% CH₃CN/5% TEAA. The flow rate was 1 mL/min. with UV detection at 260 nm. After collection and evaporation of the buffer, the DMT was cleaved from the oligonucleotides by treatment with 80% acetic acid for 30 min at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by analytical reverse phase HPLC.

The capture sequences employed in the assay for the MRSA gene are shown in Table 1 below. The detection probe oligonucleotides designed to detect MRSA genes comprise a steroid disulfide linker at the 5′-end followed by the recognition sequence. The sequences for the probes are also shown in Table 1 below.

TABLE 1

| | |
|---|---|
| | SEQ |
| | ID |
| Sequence | NO: |

| | | |
|---|---|---|
| Capture | | |
| Probe | | |
| PVRII-1 | 5′ GCCTCTGCGTATCAGTTAATGATGA-3′ | 1 |
| PVRII-2 | 5′-TATCAGTTAATGATGAGGTTTTTTTAATTG-3′ | 2 |
| PVRII-3 | 5′-GTATCAGTTAATGATGAGGTTT-3′ | 3 |
| PVRII-4 | 5′-GCGTATCAGTTAATGA-3′ | 4 |
| PVRII-5 | 5′-TCAGTTAATGATGAGG-3′ | 5 |
| PVRIII-6 | 5′-TACGCTTCTGCTTATCAGTTGATGA-3′ | 6 |
| PVRIII-7 | 5′-ATACGCTTCTGCTTATCAGTTGATGATGC-3′ | 7 |
| PVRIII-8 | 5′-CTTCTGCTTATCAGT-3′ | 8 |
| PVRIII-9 | 5′-CAGTTGATGATGCGGTT-3′ | 9 |
| PVRIII-10 | 5′-CAGTTGATGATGCGGTTTTTAA-3′ | 10 |
| Detector | | |
| Probe | | |
| NanoRR1 | TTTTAGTTTTACTTATGAT | 11 |
| NanoRR2 | ATGTCCACCATTTAACACCCTCCAA | 12 |
| NanoRR3 | ATGTCCACCATTTAACACCCT | 13 |
| NanoRR4 | AACACCCTCCAAATTATTATCTCCTCA | 14 |
| NanoRR5 | GTCACAAGGTAAAAAACTCCTCCGTTAC | 15 |
| NanoRR6 | TAAGTCACAAGGTAAAAAACTCCTCCGTTAC | 16 |
| NanoRR7 | CTTTATGATAAGTCACAAG | 17 |
| NanoRR8 | ACTCCTCCGTTACTTA | 18 |
| NanoRR9 | GATAAGTCACAAGGTAAAAA | 19 |
| NanoRR10 | ACTCCTCCGTTACTTATGATACGAT | 20 |
| NanoRR11 | TTACTTATGATACGCC | 21 |
| NanoRR12 | AACACCCTCCAAATTATTATCTC | 22 |
| NanoRR13 | TTATGATAAGTCACAAG | 23 |

The synthesis of the probe oligonucleotides followed the methods described for the capture probes with the following modifications. First, instead of the amino-modifier columns, supports with the appropriate nucleotides reflecting the 3′-end of the recognition sequence were employed. Second, the 5′-terminal steroid-cyclic disulfide was introduced in a coupling step by employing a modified phosphoramidite containing the steroid disulfide (see Letsinger et al., 2000, Bioconjugate Chem. 11:289-291 and PCT/US01/01190 (Nanosphere, Inc.), the disclosure of which is incorporated by reference in its entirety). The phosphoramidite reagent may be prepared as follows: a solution of epiandrosterone (0.5 g), 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the residue taken up in ethyl acetate. This solution was washed with water, dried over sodium sulfate, and concentrated to a syrupy residue, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. Recrystallization from pentane/ether afforded a white powder, mp 110-112° C.; ¹H NMR, δ 3.6 (1H, C³OH), 3.54-3.39 (2H, m 2OCH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in the minimum amount of dichloromethane, precipitated at −70° C. by addition of hexane, and dried under vacuum; yield 100 mg; ³¹P NMR 146.02. After completion of the DNA synthesis, the epiandrosterone-disulfide linked oligonucleotides were deprotected from the support under aqueous ammonia conditions and purified on HPLC using reverse phase column as described above.

(c) Attachment of Oligonucleotides to Gold Nanoparticles

The probe was prepared by incubating initially a 4 μM solution of the oligonucleotide with a ˜14 nM solution of a 15 nm citrate-stabilized gold nanoparticle colloid solution in a final volume of 2 mL for 24 h. The salt concentration in this preparation was raised gradually to 0.8 M over a period of 40 h at room temperature. The resulting solution was passed through a 0.2 μm cellulose acetate filter and the nanoparticle probe was pelleted by spinning at 13,000 G for 20 min. After removing the supernatant, the pellet was re-suspended in water. In a final step, the probe solution was pelleted again and resuspended in a probe storage buffer (10 mM phos, 100 mM NaCl, 0.01% w/v NaN₃). The concentration was adjusted to 10 nM after estimating the concentration based on the absorbance at 520 nm (ε=2.4×10⁸ M⁻¹cm⁻¹). The following nanoparticle-oligonucleotide conjugates specific for MRSA DNA were prepared such that the gold nanoparticle was conjugated to the 5′ end of the appropriate oligonucleotide via an epiandrosterone disulfide group.

(d) Preparation of DNA Microarrays

Arrays were printed on either NoAb (NoAb Biodiscoveries, Mississauga, Ontario) or CodeLink (Amersham Biosciences, Piscataway, N.J.) modified microscope slides using a Genomic Solutions Prosys Gantry (Genomic Solutions, Ann Arbor, Mich.) with either SynQuad non-contact dispensing nozzles or Telechem Stealth SMP3 (Telechem International, Sunnyvale, Calif.) split pins. Each spot on each array ranged from 200-400 μm in diameter, after printing. Regardless of slide type or dispensing method, amine-modified oligonucleotides were suspended in 150 mM Sodium Phosphate pH 8.5 at approximately 100 μM. Slides were arrayed at low humidity (relative humidity <30%) and subsequently rehydrated in a humidity chamber (relative humidity >70%) for approximately 18 hrs. Slides were then dried, washed to remove excess oligonucleotides, and stored in a cabinet desiccator (relative humidity <20%) until use. The positioning of the arrayed spots was designed to allow multiple hybridization experiments on each slide, achieved by partitioning the slide into separate test wells by using methods described in U.S. patent application Ser. No. 10/352,714, filed Apr. 21, 2003, which is incorporated by reference in its entirety. Each of the captures was spotted in triplicate. Protocols recommended by the manufacturer were followed for post-array processing of the slides.

(e) Hybridization

MRSA Detection Assay Procedure

The MRSA detection was performed by employing the protocol as generally described in U.S. patent application Ser. No. 10/735,357, filed Dec. 12, 2003, which is incorporated by reference in its entirety. Specifically, the MRSA assay procedure was conducted as follows. Sonicated purified genomic DNA from each bacterial sample was first denatured at 95° C. for 90 seconds and then hybridized for 30 minutes at 40° C., in a buffer containing 20% formamide, 5×SSC, 0.05% Tween 20, and a multiplex mixture of nanoparticle probes (at 250 pM), in a final volume of 50 μl. Slides were washed in 0.5 M NaNO₃, and signal developed for 3 minutes at room temperature, using a silver development solution (Nanosphere, Inc, Northbrook, Ill.). Alternatively, signal can be obtained by exposure for five minutes at room temperature to a 1:1 mixture of freshly mixed sample of the two commercial Silver Enhancer solutions (Catalog Nos. 55020 and 55145, Sigma Corporation, St. Louis, Mo.) for 5 minutes, following the Sigma protocol for the silver staining step. Slides were air-dried, and then scanned and imaged using Verigene™ (Nanosphere, Inc, Northbrook, Ill.).

### Example 2

Detection of MRSA from Bacterial Genomic DNA with Gold Nanoparticle Probes

In this Example, a method for detecting MRSA sequences using gold nanoparticle-based detection in an array format is described. Microarray plates having oligonucleotide capture probes shown in Table 1 were used along with gold nanoparticles labeled with oligonucleotides detection probes shown in Table 1. The microarray plates, capture probes, and detection probes were prepared as described in Example 1.

(a) Target DNA Preparation

Twenty-nine methicillin resistant coagulase negative (CoNS) and 19 S. aureus samples were received as swabs from Evanston Northwestern Healthcare Hospital, Evanston Hospital, Evanston, Ill. 60201. The swabs were used to inoculate a 2 ml tube of Tryptic Soy Broth (TSB) that was grown overnight at 37° C.

A loopful of the overnight culture was streaked out on (a) 5% Sheep's Blood Agar plates for individual colony growth, as well as (b) on a quadrant of a Mannitol Salt Agar plate containing 6 mcg/mL oxacillin to test for methicillin resistance. The plates were incubated for 24 hours at 37° C. Colony morphology and hemolytic patterns were recorded for each sample.

Only one sample showed colonies of mixed morphologies on blood agar. Eight samples showed colonies with mixed hemolytic patterns. Twelve samples (2 typed as CoNS and 10 typed as S. aureus) showed significant growth on oxacillin containing agar. These were designated methicillin resistant. Five samples showed very limited growth or pinpoint colonies on oxacillin containing agar, were designated methicillin semi-resistant, and were returned to 30° C. for an additional 24 hours. 31 samples showed no growth of any kind on oxacillin containing agar. These were designated methicillin sensitive.

For methicillin resistant samples, a loopful of cells representing multiple colonies was picked from the MSA-oxacillin plate and inoculated into a 2 ml tube of TSB. For methicillin semi-resistant and methicillin sensitive samples, a loopful of cells representing multiple colonies with a phenotype consistent with Staph was picked from the blood agar plate and inoculated into a 2 ml tube of TSB. The inoculated cultures were grown with shaking overnight at 37° C. then mixed with sterile glycerol and frozen at −80° C. These frozen cultures were used to inoculate TSB for growth of cells for DNA isolation. Cells were lysed using achromopeptidase, and genomic DNA was isolated using the QIAGEN Genomic DNA 20/G protocol.

(b) MRSA Gene Detection Assay

Purified genomic DNA was screened using ClearRead™ technology (Nanosphere, Inc, Northbrook, Ill.), in a microarray format, using oligonucleotides PVR 1-10 as capture probes. Briefly, 500 ng of purified genomic DNA was hybridized for 30 minutes, in a buffer containing 20% formamide, 5×SSC, 0.05% tween 20, and a multiplex mixture nanoparticle probes (NanoRR2 and NanoRR5 shown in Table 1) at 250 pM, at 40° C. (n=48 for each sample), after an initial denaturing step, as described earlier. Slides were washed in 0.5 M NaNO₃, and signal developed using silver development solution (Nanosphere, Inc, Northbrook, Ill.). Slides were scanned and imaged using Verigene™ instrument (Nanosphere, Inc, Northbrook, Ill.), and data analyzed using JMP software (SAS Institute, Inc., Cary, N.C.).

A threshold was generated using the mean intensity values+3 times the standard deviation of nine negative control spots per well. A sample was defined as giving a positive response if the intensity values were above the threshold for that sample well.

The results of the experiment are shown in Table 2. The success rate was 100%, in comparison to the results obtained from bacterial culturing; all MRSA, MSSA and MR/MS non-SA (MRCONs and MSCONs) were correctly identified. All strains which hybridized with the capture oligonucleotides PVR 1-10 and the multiplex mixture nanoparticle probes NanoRR2 and NanoRR5 (Table 1) were correctly identified as MRSA, whereas non-MRSA strains did not hybridize.

TABLE 2

| Sample Phenotypeb (from culture) | % Correct IDs | Number |
|---|---|---|
| MRSA | 100 | 8/8 |
| Non-MRSA(MSSA, MR or MS not SA) | 100 | 38/38 |

The specificity of the approach was examined by mixing methicillin resistant S. aureus (MRSA) (an example of MRCONs) genomic DNA with genomic DNA from methicillin sensitive S. aureus (MSSA). Evaluation of this mixed sample with conventional molecular biology-based approaches, such as PCR, or hybridization using a probe, using the mecA gene, should result in a false positive call, since MRSE bacteria are known to carry a copy of the mecA gene. Such a mixed sample would be indistinguishable from one that contains MRSA, if conventional techniques are utilized, resulting in a false positive for MRSA.

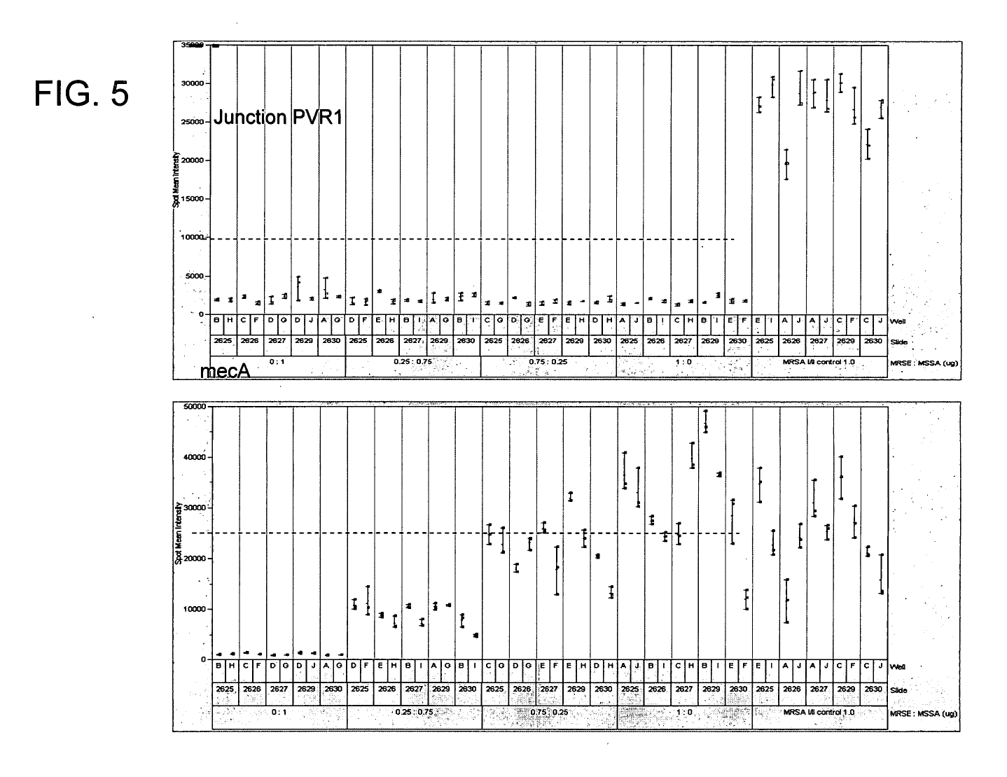

MRSE and MSSA cells were obtained from the ATCC (catalog numbers 27626 and 29213 respectively), and were cultured and genomic DNA was purified as described above. Genomic MRSE DNA was spiked into MSSA genomic DNA, with spikes ranging from of 3:1 to 1:3 (MRSE:MSSA). Microarray slides were hybridized as before, using the same probe cocktail (N=10 for each dilution). The results are shown in FIG. 5. The spiked MSSA was never mistaken for MRSA, even at the 3:1 (MRSE:MSSA) ratio. Also shown in FIG. 5 are the results obtained from a more conventional approach, where capture probes and detector probes to the mecA gene were examined in a microarray hybridization assay. The use of the mecA clearly results in mistakes, even at a 1:3 (MRSE:MSSA) ratio. The results from this experiment show the specificity of the assay.

It should be understood that the foregoing disclosure emphasizes certain specific embodiments of the invention and that all modifications or alternatives equivalent thereto are within the spirit and scope of the invention as set forth in the appended claims.

What we claim:

1. An isolated oligonucleotide consisting of:
a. a nucleic acid sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10; or
b. a nucleic acid sequence that hybridizes with the complement of the nucleic acid sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

2. A vector comprising the nucleic acid molecule of claim 1.

3. A host cell comprising the vector of claim 3.

4. A kit comprising an isolated oligonucleotide of claim 1.

5. A method for detecting methicillin resistant Staphylococcus aureus in a sample, the method comprising the steps of:
a. providing an addressable substrate having a capture probes bound thereto, the capture probes comprising an oligonucleotide of claim 1;
b. providing a detection probe comprising detector oligonucleotides, wherein the detector oligonucleotides have sequences that are complementary to at least a portion of the MRSA nucleic acid sequence;
c. contacting the sample with the substrate and the detection probe under conditions that are effective for the hybridization of the capture oligonucleotide to the MRSA nucleic acid sequence and the hybridization of the detection probe to the MRSA nucleic acid sequence;
d. washing the substrate to remove non-specifically bound material; and
e. detecting whether the capture oligonucleotide and detection probe hybridized with the MRSA nucleic acid sequence.

6. The method of claim 5, wherein the capture oligonucleotide comprises a nucleic acid sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

7. The method of claim 5, wherein the detector oligonucleotides comprise a nucleic acid sequence as set forth in SEQ ID NO: 1; SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23.

8. The method of claim 5, wherein sample is contacted with the detector probe so that methicillin resistant Staphylococcus aureus nucleic acid present in the sample hybridizes with the detector oligonucleotides on the detector probe, and the methicillin resistant Staphylococcus aureus nucleic acid bound to the detector probe is then contacted with the substrate so that the methicillin resistant Staphylococcus aureus nucleic acid hybridizes with the capture oligonucleotide on the substrate.

9. The method of claim 5, wherein sample is contacted with the substrate so that a methicillin resistant Staphylococcus aureus nucleic acid present in the sample hybridizes with a capture oligonucleotide, and the methicillin resistant Staphylococcus aureus nucleic acid bound to the capture oligonucleotide is then contacted with the detector probe so that the methicillin resistant Staphylococcus aureus nucleic acid hybridizes with the detector oligonuclotides on the detector probe.

10. The method of claim 5, wherein the sample is contacted simultaneously with the detector probe and the substrate.

11. The method of claim 5, wherein the detector oligonucleotides comprise a detectable label.

12. The method of claim 11, wherein the detectable label allows detection by photonic, electronic, acoustic, opto-acoustic, gravity, electrochemical, electro-optic, mass-spectrometric, enzymatic, chemical, biochemical, or physical means.

13. The method of claim 11, wherein the label is fluorescent.

14. The method of claim 11, wherein the label is luminescent.

15. The method of claim 11, wherein the label is phosphorescent.

16. The method of claim 11, wherein the label is radioactive.

17. The method of claim 11, wherein the label is a nanoparticle.

18. The method of claim 11, wherein the label is a dendrimer.

19. The method of claim 11, wherein the label is a molecular aggregate.

20. The method of claim 11, wherein the label is a quantum dot.

21. The method of claim 11, wherein the label is a bead.

22. The method of claim 5, wherein the detector probe is a nanoparticle probe having detector oligonucleotides bound thereto.

23. The method of claim 22, wherein the nanoparticles are made of a noble metal.

24. The method of claim 23, wherein the nanoparticles are made of gold or silver.

25. The method of claim 24, wherein the nanoparticles are made of gold.

26. The method of claim 23, wherein the detecting comprises contacting the substrate with silver stain.

27. The method of claim 23, wherein the detecting comprises detecting light scattered by the nanoparticle.

28. The method of claim 23, wherein the detecting comprises observation with an optical scanner.

29. The method of claim 28, wherein the scanner is linked to a computer loaded with software capable of calculating grayscale measurements, and the grayscale measurements are calculated to provide a quantitative measure of the amount of nucleic acid detected.

30. The method of claim 23, wherein the detecting comprises observation with a flatbed scanner.

31. The method of claim 30, wherein the scanner is linked to a computer loaded with software capable of calculating grayscale measurements, and the grayscale measurements are calculated to provide a quantitative measure of the amount of nucleic acid detected.

32. The method of claim 23, wherein the oligonucleotides attached to the substrate are located between two electrodes, the nanoparticles are made of a material that is a conductor of electricity, and step (d) comprises detecting a change in conductivity.

33. The method of claim 32, wherein the electrodes are made of gold and the nanoparticles are made of gold.

34. The method of claim 32, wherein the substrate is contacted with silver stain to produce the change in conductivity.

35. The method of claim 5, wherein the sample comprises nucleic acid molecules of higher biological complexity relative to amplified nucleic acid molecules.

36. The method of claim 35, wherein the higher biological complexity is greater than about 50,000.

37. The method of claim 35, wherein the higher biological complexity is between about 50,000 and about 3,000,000.

38. The method of claim 35, wherein the higher biological complexity is about 3,000,000.

39. The method of claim 5, wherein nucleic acid molecules in the sample are amplified.

40. The method of claim 39, wherein the nucleic acid molecules in the sample are amplified by polymerase chain reaction, rolling circle amplification, NASBA, or iCAN.

41. A method for detecting methicillin resistant Staphylococcus aureus in a sample, the method comprising the steps of:
a. providing an addressable substrate having a capture oligonucleotide bound thereto, wherein the capture probe comprises an oligonucleotide having a sequence complementary to at least a portion of the MRSA nucleic acid sequence;
b. providing a detection probe comprising detector oligonucleotides, wherein the detector oligonucleotides is an oligonucleotide of claim 1;
c. contacting the sample with the substrate and the detection probe under conditions that are effective for the hybridization of the capture oligonucleotide to the MRSA nucleic acid sequence and the hybridization of the detection probe to the MRSA nucleic acid sequence;
d. washing to the substrate to remove non-specifically bound material; and
e. detecting whether the capture oligonucleotide and detection probe hybridized with the MRSA nucleic acid sequence.

42. The method of claim 41, wherein the detector oligonucleotides comprise a nucleic acid sequence as set forth in SEQ ID NO: 1; SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

43. The method of claim 41, wherein the capture oligonucleotides comprise a nucleic acid sequence as set forth in SEQ ID NO: 1; SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23.

44. The method of claim 41, wherein sample is contacted with the detector probe so that a methicillin resistant Staphylococcus aureus nucleic acid present in the sample hybridizes with the detector oligonucleotides on the detector probe, and the methicillin resistant Staphylococcus aureus nucleic acid bound to the detector probe is then contacted with the substrate so that the methicillin resistant Staphylococcus aureus nucleic acid hybridizes with the capture oligonucleotide on the substrate.

45. The method of claim 41, wherein sample is contacted with the substrate so that a methicillin resistant Staphylococcus aureus nucleic acid present in the sample hybridizes with a capture oligonucleotide, and the methicillin resistant Staphylococcus aureus nucleic acid bound to the capture oligonucleotide is then contacted with the detector probe so that the methicillin resistant Staphylococcus aureus nucleic acid hybridizes with the detector oligonuclotides on the detector probe.

46. The method of claim 41, wherein the sample is contacted simultaneously with the detector probe and the substrate.

47. The method of claim 41, wherein the detector oligonucleotides comprise a detectable label.

48. The method of claim 47, wherein the detectable label allows detection by photonic, electronic, acoustic, opto-acoustic, gravity, electrochemical, electro-optic, mass-spectrometric, enzymatic, chemical, biochemical, or physical means.

49. The method of claim 47, wherein the label is fluorescent.

50. The method of claim 47, wherein the label is luminescent.

51. The method of claim 47, wherein the label is phosphorescent.

52. The method of claim 47, wherein the label is radioactive.

53. The method of claim 47, wherein the label is a nanoparticle.

54. The method of claim 47, wherein the label is a dendrimer.

55. The method of claim 47, wherein the label is a molecular aggregate.

56. The method of claim 47, wherein the label is a quantum dot.

57. The method of claim 47, wherein the label is a bead.

58. The method of claim 41, wherein the detector probe is a nanoparticle probe having detector oligonucleotides bound thereto.

59. The method of claim 58, wherein the nanoparticles are made of a noble metal.

60. The method of claim 59, wherein the nanoparticles are made of gold or silver.

61. The method of claim 60, wherein the nanoparticles are made of gold.

62. The method of claim 58, wherein the detecting comprises contacting the substrate with silver stain.

63. The method of claim 58, wherein the detecting comprises detecting light scattered by the nanoparticle.

64. The method of claim 58, wherein the detecting comprises observation with an optical scanner.

65. The method of claim 64, wherein the scanner is linked to a computer loaded with software capable of calculating grayscale measurements, and the grayscale measurements are calculated to provide a quantitative measure of the amount of nucleic acid detected.

66. The method of claim 58, wherein the detecting comprises observation with a flatbed scanner.

67. The method of claim 66, wherein the scanner is linked to a computer loaded with software capable of calculating grayscale measurements, and the grayscale measurements are calculated to provide a quantitative measure of the amount of nucleic acid detected.

68. The method of claim 58, wherein the oligonucleotides attached to the substrate are located between two electrodes, the nanoparticles are made of a material that is a conductor of electricity, and step (d) comprises detecting a change in conductivity.

69. The method of claim 68, wherein the electrodes are made of gold and the nanoparticles are made of gold.

70. The method of claim 68, wherein the substrate is contacted with silver stain to produce the change in conductivity.

71. The method of claim 41, wherein the sample comprises nucleic acid molecules of higher biological complexity relative to amplified nucleic acid molecules.

72. The method of claim 66, wherein the higher biological complexity is greater than about 50,000.

73. The method of claim 66, wherein the higher biological complexity is between about 50,000 and about 3,000,000.

74. The method of claim 66, wherein the higher biological complexity is about 3,000,000.

75. The method of claim 41, wherein nucleic acid molecules in the sample are amplified.

76. The method of claim 41, wherein the nucleic acid molecules in the sample are amplified by polymerase chain reaction, rolling circle amplification, NASBA, or iCAN.

77. The method of claims 1 or 41, wherein the capture probe and substrate are bound by specific binding pair interactions.

78. The method of claim 77 wherein the capture probe and substrate comprise complements of a specific binding pair.

79. The method of claim 78 wherein complements of a specific binding pair comprise nucleic acid, oligonucleotide, peptide nucleic acid, polypeptide, antibody, antigen, carbohydrate, protein, peptide, amino acid, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances.


SEQUENCE LISTING (23 sequences)

SEQ ID NO: 1 - DNA, 25 bp, Artificial Sequence
  Synthetic sequence that is a capture probe for the MRSA gene.
gcctctgcgtatcagttaatgatga

SEQ ID NO: 2 - DNA, 30 bp, Artificial Sequence
  Synthetic sequence that is a capture probe for the MRSA gene.
tatcagttaatgatgaggtttttttaattg

SEQ ID NO: 3 - DNA, 22 bp, Artificial Sequence
  Synthetic sequence that is a capture probe for the MRSA gene.
gtatcagttaatgatgaggttt

SEQ ID NO: 4 - DNA, 16 bp, Artificial Sequence
  Synthetic sequence that is a capture probe for the MRSA gene.
gcgtatcagttaatga

SEQ ID NO: 5 - DNA, 16 bp, Artificial Sequence
  Synthetic sequence that is a capture probe for the MRSA gene.
tcagttaatgatgagg

SEQ ID NO: 6 - DNA, 25 bp, Artificial Sequence
  Synthetic sequence that is a capture probe for the MRSA gene.
tacgcttctgcttatcagttgatga

SEQ ID NO: 7 - DNA, 29 bp, Artificial Sequence
  Synthetic sequence that is a capture probe for the MRSA gene.
atacgcttctgcttatcagttgatgatgc

SEQ ID NO: 8 - DNA, 15 bp, Artificial Sequence
  Synthetic sequence that is a capture probe for the MRSA gene.
cttctgcttatcagt

SEQ ID NO: 9 - DNA, 17 bp, Artificial Sequence
  Synthetic sequence that is a capture probe for the MRSA gene.
cagttgatgatgcggtt

SEQ ID NO: 10 - DNA, 22 bp, Artificial Sequence
  Synthetic sequence that is a capture probe for the MRSA gene.
cagttgatgatgcggtttttaa

SEQ ID NO: 11 - DNA, 19 bp, Artificial Sequence
  Synthetic sequence that is a detector probe for the MRSA gene.
ttttagttttacttatgat

SEQ ID NO: 12 - DNA, 25 bp, Artificial Sequence
  Synthetic sequence that is a detector probe for the MRSA gene.
atgtccaccatttaacaccctccaa

SEQ ID NO: 13 - DNA, 21 bp, Artificial Sequence
  Synthetic sequence that is a detector probe for the MRSA gene.
atgtccaccatttaacaccct

SEQ ID NO: 14 - DNA, 27 bp, Artificial Sequence
  Synthetic sequence that is a detector probe for the MRSA gene.
aacaccctccaaattattatctcctca

SEQ ID NO: 15 - DNA, 28 bp, Artificial Sequence
  Synthetic sequence that is a detector probe for the MRSA gene.
gtcacaaggtaaaaaactcctccgttac

SEQ ID NO: 16 - DNA, 31 bp, Artificial Sequence
  Synthetic sequence that is a detector probe for the MRSA gene.
taagtcacaaggtaaaaaactcctccgttac

SEQ ID NO: 17 - DNA, 19 bp, Artificial Sequence
  Synthetic sequence that is a detector probe for the MRSA gene.
ctttatgataagtcacaag

SEQ ID NO: 18 - DNA, 16 bp, Artificial Sequence
  Synthetic sequence that is a detector probe for the MRSA gene.
actcctccgttactta

SEQ ID NO: 19 - DNA, 20 bp, Artificial Sequence
  Synthetic sequence that is a detector probe for the MRSA gene.
gataagtcacaaggtaaaaa

SEQ ID NO: 20 - DNA, 25 bp, Artificial Sequence
  Synthetic sequence that is a detector probe for the MRSA gene.
actcctccgttacttatgatacgat

SEQ ID NO: 21 - DNA, 16 bp, Artificial Sequence
  Synthetic sequence that is a detector probe for the MRSA gene.
ttacttatgatacgcc

SEQ ID NO: 22 - DNA, 23 bp, Artificial Sequence
  Synthetic sequence that is a detector probe for the MRSA gene.
aacaccctccaaattattatctc

SEQ ID NO: 23 - DNA, 17 bp, Artificial Sequence
  Synthetic sequence that is a detector probe for the MRSA gene.
ttatgataagtcacaag